(19) United States
(12) Patent Application Publication
(10) Pub. No.: US 2020/0214780 A1
(43) Pub. Date: Jul. 9, 2020

(54) Apparatus and Method for Manufacturing Medical and Dental Cabinets

(71) Applicants: Jeremy Bedwell, Tulsa, OK (US); David Riesen, Tulsa, OK (US)

(72) Inventors: Jeremy Bedwell, Tulsa, OK (US); David Riesen, Tulsa, OK (US)

(21) Appl. No.: 16/734,266

(22) Filed: Jan. 3, 2020

Related U.S. Application Data
(60) Provisional application No. 62/788,099, filed on Jan. 3, 2019.

(51) Int. Cl. A61B 50/10; G06F 30/20

(52) U.S. Cl. CPC A61B 50/10; A47B 2210/00; A61B 2050/105; G06F 30/20

(57) ABSTRACT

The method of construction of medical and dental cabinets of the present invention enables the user to construct cabinets that are better suited for such environments through the use of PVC board substrate that is either wrapped with a vinyl wrapping or high pressure laminate material.

### CROSS-REFERENCE TO RELATED APPLICATIONS

This application claims the priority of U.S. Provisional Application No. 62/788,099 filed Jan. 3, 2019.

### STATEMENT REGARDING FEDERALLY SPONSORED RESEARCH OR DEVELOPMENT

Not Applicable.

### BACKGROUND

The present invention relates to cabinets used in the medical and dental fields and more particularly to improved medical and dental cabinets that are lightweight, sterile, water/liquid-resistant, stronger, easier to install, and compliant with safety and health regulations when compared to prior art cabinets used in the medical and dental fields.

Typical cabinets used in the offices of medical and dental industries have many disadvantages. Many of these disadvantages stem from the specific materials used in the manufacture of such cabinets. These types of cabinets are typically manufactured using wood, plywood, particleboard, or medium density fiberboard (“MDF”) as the substrate. The substrate or exterior of the cabinets are typically covered with either laminate or melamine. Some prior art cabinets are constructed of metal, which brings its own list of disadvantages. Prior art cabinets typically include a metal base on which the actual cabinets sit, so that the cabinets are kept off of direct contact with the floor. While this might sound like an advantage initially, the reason the metal base is required in the first place is due to the porous quality of the substrates and therefore its ability to absorb liquids. The medical and dental environments typically include the use of liquids, thereby requiring that the cabinets be kept off of direct contact with the floor.

Prior art cabinets are typically made from wood, plywood, particleboard, or medium density fiberboard (“MDF”). The cabinets are then typically covered with a laminate or melamine to make the cabinets aesthetically pleasing. In some instances, metal/steel is used to construct the cabinets. There are inherent qualities in all of these materials typically used for medical and dental cabinets that create a need for a new and novel cabinet and method of manufacturing cabinets for such use.

Most of the cabinets currently manufactured for use in medical and dental environments typically use MDF as the substrate material. MDF is an engineered wood composite material made up of wood fibers that are combined using wax and resin and then heat pressed together. MDF is typically heavier than other substrate materials, such as plywood and particleboard. While there are advantages for using MDF over the other typical substrate options, MDF has plenty of disadvantages for use in medical and dental environments. First, MDF is essentially glorified particle board, and just like particle board, MDF will soak up water and other liquids like a sponge, which causes it to swell and break down. Due to the makeup of MDF, problems also arise with attaching components and hardware using screws because MDF generally does not hold screws well due to the fine particles/wood fibers. Attaching components and hardware using screws can cause MDF to split, break down, and otherwise deteriorate. Prior art MDF cabinets that are prominent in the dental and medical vertical markets typically include several open and closed storage compartments that must be required to hold equipment weight and day to day abuse without losing its integrity. This is an area of concern in which MDF cabinets fail to provide the ideal solution. Once the integrity of the MDF cabinet has been compromised due to water, screws, hardware, or has otherwise acquired any physical damage, the MDF will continue to deteriorate and become a larger issue throughout the entire cabinet.

Prior art cabinets are also sometimes manufactured using plywood. Plywood, like MDF, is also susceptible to water damage. Plywood also has issues with rough edges, splintering and chipping when being cut to size or screwed into. Plywood as a cabinet substrate material is also heavy, although typically not as heavy as MDF cabinets.

Due to the potential for water damage associated with the substrate materials typically used in prior art cabinets, the cabinets typically have to be set up off of the ground. This is typically achieved by using a metal subbase that the cabinets themselves sit on top of. Electrical wiring and other accessories are sometimes run through this metal subbase as well. The metal subbases of the cabinets are designed to keep the cabinets of MDF construction off the floor to avoid as much water/liquid damage as possible. However, the metal subbase brings its own list of issues as well, such as: bacteria build up in the seams, sharp edges, denting and limited access to the lower structure of the cabinets. Further, the metal subbase is subject to rusting if and when it comes into contact with water or other liquid. There is almost always water or other liquids being used in the offices of medical and dental industries. Use of a subbase is also less aesthetically pleasing than if the cabinets were able to sit flush with the floor.

With prior art cabinets, the substrate material is typically covered with a laminate material or a melamine material to make the cabinets aesthetically pleasing and offer some protection for the MDF or plywood from the elements. However, there are also several drawbacks to these materials. Melamine can be prone to chipping and splintering. Melamine type cabinets are also usually heavier than other cabinets. Laminate shares some of the same drawbacks as melamine and in damp or wet environments, laminate will eventually begin to crack, peel or discolor. Once these destructive actions begin, they continue to get worse daily and once the MDF under the laminate starts to crumble or show signs of abuse, the MDF material is virtually impossible to repair and the entire section of the cabinet must be replaced.

Some other prior art cabinets are constructed of metal. In the medical and dental environments where there is often water or liquids present, this obviously creates rust and corrosion problems. More disadvantages associated with metal cabinets include, sharp and unsanitary edges, denting, poor integrity throughout and heavy because typically made from a large gauge of sheet metal.

Prior art cabinets are typically constructed of pieces of varying thicknesses, which makes the manufacturing process cumbersome. For example, the back panels are typically manufactured using a thinner material than the other panels. Further, due to the use of varying thicknesses, prior art cabinets typically require internal strengthening components such as metal frames or chassis to make up for weak parts of the cabinets due to thinner materials being used.

Of extreme importance in medical and dental environments is proper sterilization. Both MDF and plywood are porous and therefore susceptible to not only absorbing liquids, but also bacteria. In the medical and dental environments, sterilization is one of the most important requirements. In fact, in the dental field, dentists have to comply with certain rules and regulations, including regulations from the Occupational Safety and Health Administration (“OSHA”). Even with the laminate or melamine material covering the MDF or plywood, sterilization of the prior art cabinets can be difficult. This is because many of the products used for sterilization in these environments can cause the laminate or melamine materials to break down. Most laminate or melamine materials are not resistant to many of the cleaners, disinfectants, and other chemicals used to sterilize these environments. Therefore, the process of sterilization that must be used with the prior art cabinets is made more difficult than it could be, i.e., if these problems did not exist, a simple way of sterilizing would be through the use of a Steri-Wipe or similar product that complies with rules and regulations.

Medical and dental environments must maintain strict sterilization guidelines and use industry standard cleaning and sterilization products. One of the issues with prior art cabinets that is consistently run into is that the cleaning material is a liquid based material that can break down the integrity of cabinetry. Examples of this are chemicals in the cleaners can break down the glue that has bonded the laminate or edge banding to the MDF. Not only does this start a negative chain of events for the exterior of the cabinets but it also allows access for liquids to penetrate into the MDF board.

Due to the unique environment where medical and dental cabinets are used, and the various aforementioned drawbacks inherent in the prior art cabinets currently used, there is a need for a new and improved cabinet for use in medical and dental environments and a method of manufacturing such a cabinet. For the foregoing reasons, there is a need for a new and improved cabinet and method of manufacturing the cabinet that is lightweight, sterile, water/liquid-resistant, stronger, easier to install, compliant with safety and health regulations, and better suited for use in medical and dental environments when compared to prior art cabinets.

### SUMMARY

The present invention is directed to a new and improved cabinet for use in medical and dental environments and method of manufacturing the cabinet that overcomes the disadvantages found in the prior art.

Accordingly, it is an object of the present invention to provide a new and improved cabinet for medical and dental environments and a method of manufacturing the cabinet that overcomes the disadvantages found in the prior art cabinets due to the materials that prior art cabinets are manufactured from.

The present invention includes elements of cabinet construction, and application of vinyl wrap material and/or laminate material to produce a novel cabinet for use in the medical and dental environments.

In a preferred embodiment, a cabinet of the present invention is comprised of a substrate of solid-core PVC board which is then covered with an adhesive vinyl wrapping material.

In another embodiment of the present invention, a cabinet of the present invention is comprised of the solid-core PVC board substrate and is covered with a high pressure laminate material (“HPL”).

Solid-core PVC board is typically used for large signage and displays for promotional advertisements, both indoor and outdoor. Solid-core PVC board has many benefits as a material for various uses, but specifically for use as a cabinet substrate when used as it is in the present invention. Some of the benefits include: relatively lightweight and easy to handle, easy to fabricate, chemical and corrosion resistance, moisture resistance, low water absorption, low flammability, bonds easily to various substrates, comes in wide range of thicknesses, great for indoor or outdoor use, weather resistant, smooth exterior, non-porous, very durable, accepts and holds screws well, rigid, high impact strength, high tensile strength and hardness.

Solid-core PVC board has not typically been used as cabinet substrate material because it does have certain drawbacks for use as cabinetry. Solid-core PVC board typically only comes in a very limited number of colors and those colors are typically base colors such as white, black, gray. More importantly, solid-core PVC board shows imperfections relatively easily, such as scratches, screw holes, and other imperfections that make it not very aesthetically pleasing for use as a cabinet. However, the present invention overcomes these drawbacks through the use of the vinyl wrapping material and/or laminate material to cover the solid-core PVC substrate and therefore provides an improved cabinet for use in the medical and dental environments that has many benefits over prior art cabinets.

The present invention uses solid-core PVC board as the substrate material because of the many advantageous properties it provides for use in medical and dental environments over prior art substrate materials such as wood, plywood, particleboard, or MDF. Since the solid-core PCV board is water resistant, it is not susceptible to water damage like the prior art substrates, which is of extreme importance for use in the medical and dental environments. Due to this, a cabinet of the present invention does not require a metal base and can sit flush on the ground, or it can use a toe kick made of the same substrate material rather than metal. This property of water resistance also provides better protection against bacteria when compared to the prior art because it will not soak up liquids.

Solid-core PVC board is also better suited for medical and dental environments than the prior art materials, because it is more durable, holds screws better the prior art substrates, and can also be bonded using most adhesives, including wood glue. These properties also allow for different types of anchors and mounts to be used on cabinets of the present invention which provides for easier installation. Solid-core PCV board material also generally weighs less when compared to the prior art substrate materials, therefore producing a lighter cabinet than prior art cabinets. Solid-core PVC board material also has low flammability, and good surface resistance, tracking resistance, and arc resistance when compared to prior are materials.

Further, the present invention combines the solid-core PVC board substrate with either a vinyl wrap material or a laminate material that covers the outside of the substrate and provides additional benefits for use in the harsh medical and dental environments.

Thus, a principal object of the present invention is to provide an improved cabinet for use in medical and dental environments that eliminates the issues with prior art cabinets used in these environments.

Another object of the present invention is to provide a method of manufacturing an improved cabinet for use in medical and dental environments.

Yet another object of the present invention is to provide a cabinet that is easily sterilized without sacrificing the integrity of the cabinet.

Another object of the present invention is to provide a cabinet that can be sterilized using OSHA or other regulatory body required/approved sterilization procedures, such as use of Steri-wipes or similar products.

Another object of the present invention is to provide a cabinet that can withstand the harsh environments found in medical and dental offices.

Yet another object of the present invention is to provide a cabinet that is water and/or liquid resistant.

A further object of the present invention is to provide a cabinet that is more durable and stronger than prior art cabinets.

A further object of the present invention is to provide a cabinet that is easier to install than prior art cabinets.

While it is to be understood that both the foregoing general description and the following detailed description are exemplary and explanatory only and are not restrictive on the present invention, these and other features, aspects, and advantages of the present invention will become better understood with regard to the following detailed description and accompanying drawings where:

### BRIEF DESCRIPTION OF THE DRAWINGS

FIG. 1 is a process flow diagram for a medical and/or dental cabinet apparatus and method of construction with one exemplary embodiment of the present invention;

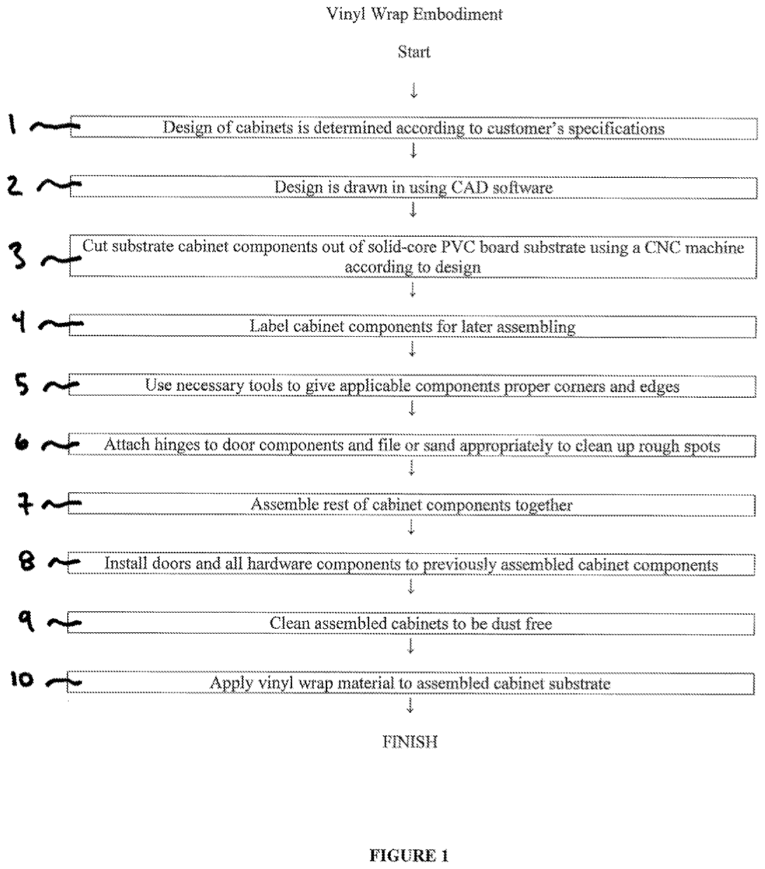

FIG. 2 is a process flow diagram for a medical and/or dental cabinet apparatus and method of construction with a second embodiment of the present invention;

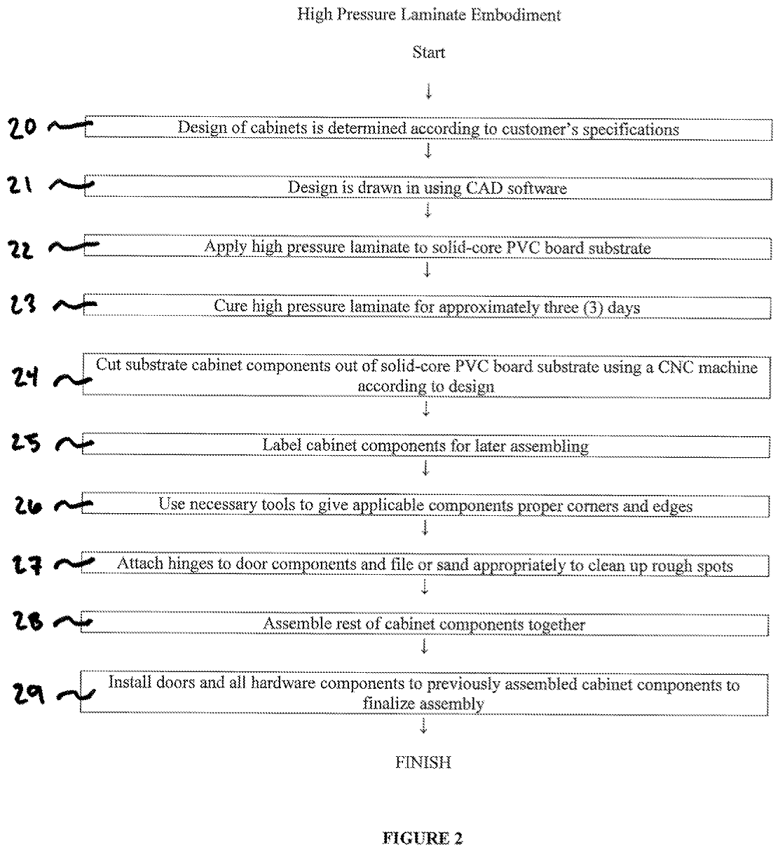

### DESCRIPTION

Referring now to the drawings, reference will be made to the preferred embodiment of the invention, an example of which is illustrated in the accompanying drawings and not meant for purposes of limited the scope of the present invention.

The method of manufacturing the cabinets of the present invention vary slightly dependent on whether the substrate is covered using a vinyl wrap or a high pressure laminate material. In one embodiment of the present invention, a method of manufacturing a cabinet with a solid-core PVC board substrate and a vinyl wrap exterior may be summarized as follows. The design pattern of the cabinet components is determined according to customer specifications; whether, comprising upper and/or lower cabinets, modularity, free standing and/or wall mounted, pass-through, storage configurations, and workflow designs (Step 1). Once determined, the cabinet components are drawn using CAD software on a computer (Step 2). The size of solid-core PVC board substrate that is typically used is a 4 foot by 8 foot, ¾″ thick sheet. Although, larger or smaller sizes and thicknesses may be used depending on the application. Then, a CNC machine is used to cut the substrate into the cabinet components according to the design (Step 3). The cabinet components are labeled (Step 4) and then tools are used to give the applicable components the proper corners and edges (Step 5). The cabinet components that make up the doors are then hinged and filed or sanded appropriately to clean up any rough spots (Step 6). The rest of the cabinet components are then assembled together (Step 7). Then the doors and all hardware components are installed (step 8) and the cabinets are cleaned so that they are dust free (step 9).

In the embodiment described in the preceding paragraph, once the cabinets have been assembled, then a vinyl wrap material is applied to the substrate to make the cabinets aesthetically pleasing and provide extra protection for the medical and dental environments (Step 10). In this embodiment, the preferred vinyl wrap material is a material manufactured by 3M™, called 3M™ DI-NOC™ Architectural Finishes, although any vinyl wrap material having similar characteristics may be used. The use of a vinyl wrap to cover the substrate material provides many benefits for use in medical and dental environments. First, vinyl wrap allows for a large amount of choices to the customer of different looks and finishes. Second, the vinyl wrap materials used for cabinets of the present invention share some of the same benefits as the solid-core PVC board substrate, namely, that they are water/moisture resistant, heat resistant, fire o10 resistant, and resistant to most solvents, cleaners, and chemicals used in cleaning and sterilization.

An important distinction of the present invention over the prior art is that the vinyl wrap material requires a smooth, clean, non-porous surface to properly adhere to the substrate. Therefore, prior art substrate materials such as MDF, plywood, wood, and particleboard are not good materials to be used with the vinyl wrap material as it will not adhere properly to such substrates. However, the solid-core PVC board used for the cabinets of the present invention is an ideal material that provides the smooth high surface tension area needed to proper adhesion of the vinyl wrap.

In another embodiment of the present invention, a method of manufacturing a cabinet with a solid-core PVC board substrate and high-pressure laminate (“HPL”) exterior may be summarized as follows. The steps of manufacturing the cabinets are essentially the same as the method in the preceding embodiment, with the exception that that high-pressure laminate is applied to the solid-core PVC board sheets prior to cutting them into the components using the CNC machine; i.e. the HPL is applied prior to construction of the cabinets versus in the other embodiment the vinyl wrap material is applied after the construction of the cabinets. The HPL replaces the use of the vinyl wrap material in the previous embodiment. First, the design of the cabinet components is determined according to customer specifications (step 20). Once determined, the cabinet components are drawn using CAD software on a computer (step 21). The HPL is applied to the solid-core PVC board substrate sheets (step 22) and allowed to cure/set for approximately three (3) days (step 23). Once the HPL has properly cured, the manufacturing process proceeds as previously described and the cabinets are assembled. The cabinet components are cut out of the HPL covered solid-core PVC board using a CNC machine (step 24). The cabinet components are labelled for later assembling (step 25). Necessary tools are used to give applicable components proper corners and edges according to the design (step 26). Attach hinges to door components and file/sand appropriately to clean up any rough spots (step 27). The rest of the cabinet components, minus the door components, are then all assembled together (step 28). Finally, the doors and all hardware components are assembled with the previously assembled components to finalize assemble (step 29). As mentioned, the HPL takes the place of the vinyl wrap material in this embodiment, so that step of applying the vinyl wrap is eliminated under this embodiment. In this specific embodiment, the preferred HPL comes from Wilsonart® HPL, although any HPL materials sharing similar beneficial characteristics may be used.

An important feature of the present invention is that the cabinets are constructed using the same substrate thickness for all components, for example a ¾″ thick solid-core PVC board can be used for the doors, side panels, and back panels. This feature results in the cabinets of the present invention being stronger than prior art cabinets, which typically use varying thicknesses throughout, especially for the back panels. This feature also results in cabinets of the present invention not requiring the use of internal strengthening components typically found in the prior art. The solid-core PVC substrate provides an overall stronger cabinet that can withstand the medical and dental environments without the need for such internal chassis or strengthening components. Further, by using the same substrate thickness throughout, the manufacturing process is less cumbersome than for prior art cabinets.

The cabinet of the present invention also provides benefits over prior art cabinets during installation by making installation easier in several areas and/or allowing for certain things to be done during installation that could not be performed with prior art cabinets. Since the solid-core PVC substrate of the present invention provides a lighter overall cabinet than prior art cabinets, the cabinets of the present invention are easier for installers to maneuver and install. This is especially beneficial when the cabinet installation requires the cabinets to be hung or lifted. Many times during installation of cabinets, adjustments are required to be made due to a variety of potential reasons not initially known, such as an uneven floor, unforeseen errors, and even construction imperfections. These adjustments offer require the repositioning of screws and/or other attachment means. As has been discussed herein, prior art cabinets, specifically MDF cabinets, do not hold screws well, especially if adjustments must be made and components and hardware must be repositioned and re-attached in a new screw hole. These adjustments, which are common when installing cabinets, can cause the prior art cabinets to lose strength and overall integrity. Often times during the installation, other adjustments or alterations will be required due to electrical or plumbing access being installed in an incorrect location or otherwise not as originally designed. With prior art cabinets, specifically MDF cabinets, the cabinet is only as strong as the laminate surrounding the substrate. Therefore, in instances where the electrical or plumbing access is incorrectly located, it is typically not recommended to cut/drill new access to fit the environment provided, as doing so will decrease the integrity of the cabinet. However, the cabinet of the present invention overcomes these issues because the solid-core PVC substrate does not share the inherent drawbacks found in the prior art. All of these adjustments can be handled by the cabinet of the present invention without sacrificing the strength and integrity of the cabinet, which is a huge benefit during installation as it avoids potential setbacks. Further, the PVC substrate of the cabinet of the present invention allows for electrical components to be added on to the cabinets in the field without compromising the integrity of the cabinet as a whole.

Also, since the cabinet of the present invention does not require the use of a metal subbase like the prior art cabinets, the cabinet of the present invention can be installed to sit flush with the floor as a solid one piece cabinet, using detachable toe-kicks or other means of attaching the cabinet to the floor. When installing into a wall, since the substrate used in the cabinet of the present invention is comprised of the same thickness for all components, including the back panel, there is unlimited area to attach the cabinet using trim screws or other attachment means.

It is within the ambit of the present invention to cover any obvious modifications of the embodiments described herein.

What is claimed is:

1. A method of constructing a cabinet for medical and dental environments comprising:
determining specific design of a cabinet according to a customer's specifications;
drawing said design using CAD (computer aided design) software;
applying a high pressure laminate to a solid-core PVC substrate;
curing the high pressure laminate for approximately three (3) days;
cutting the cabinet components out of the solid-core PVC substrate using a CNC (computer numerical control) machine;
labeling the cabinet components for ease of later assembly;
preparing applicable cabinet components by using tools to give proper corners and edges required per the design;
attaching hinges to door cabinet components;
assembling remaining cabinet components; and
installing door cabinet components and any hardware components to the remaining assembled cabinet components to finalize assembly of cabinet.

2. The method of claim 1 wherein said PVC substrate is a 4 foot by 8 foot, ¾″ thick sheet.

3. The method of claim 1 wherein said high pressure laminate is Wilsonart® high pressure laminate.

4. A method of constructing a cabinet for medical and dental environments comprising:
determining specific design of a cabinet according to a customer's specifications;
drawing said design using CAD (computer aided design) software;
cutting the cabinet components out of the solid-core PVC substrate using a CNC (computer numerical control) machine;
labeling the cabinet components for ease of later assembly;
preparing applicable cabinet components by using tools to give proper corners and edges required per the design;
attaching hinges to door cabinet components;
assembling remaining cabinet components;
installing door cabinet components and any hardware components to the remaining assembled cabinet components to finalize assembly of cabinet;
cleaning assembled cabinet to be dust free; and
applying a vinyl wrap material to the assembled cabinet substrate.

5. The method of claim 4 wherein said PVC substrate is a 4 foot by 8 foot, ¾″ thick sheet.

6. The method of claim 4 wherein said vinyl wrap material is 3M™ DI-NOC™ Architectural Finishes.

7. A cabinet for use in medical and dental environments comprising:
a substrate material made out of solid-core PVC;
a high pressure laminate applied to the substrate.

8. A cabinet for use in medical and dental environments comprising:
a substrate material made out of solid-core PVC;
a vinyl wrap material applied to the substrate.
